(19) United States
(12) Patent Application Publication
(10) Pub. No.: US 2015/0075005 A1
(43) Pub. Date: Mar. 19, 2015

(54) SURGICAL ROD CUTTER

(71) Applicant: Aesculap AG, Tuttlingen (DE)

(72) Inventors: Stephan Lindner, Wurmlingen (DE); Christoph Burmeister, Singen am Hohentwiel (DE)

(21) Appl. No.: 14/486,155

(22) Filed: Sep. 15, 2014

(30) Foreign Application Priority Data
Sep. 16, 2013 (DE) 10 2013 110 184.4

(51) Int. Cl. A61B 17/88

(52) U.S. Cl. CPC A61B 17/8863
USPC 30/99; 30/95

(57) ABSTRACT

A surgical rod cutter includes first and second device parts adapted to be rotated relative to one another about an axis of rotation. The device parts each have a continuous opening for accommodating a rod to be severed, the opening having its longitudinal axis oriented in the direction of the axis of rotation and a cutting edge. The cutting edge of the second device part is formed in opposed relationship with the cutting edge of the first device part. The openings are arranged with the same amount of eccentricity with respect to the axis of rotation. The device parts are operatively connected to one another by teeth and can be positioned relative to one another. At least one of the openings can have replaceably arranged therein a bushing provided with the cutting edge.

### RELATED APPLICATIONS

This application is related to, and claims the benefit of priority under 35 U.S.C. §119 of, German Application No. DE 10 2013 110 184.4, filed Sep. 16, 2013, the content of which is incorporated by reference herein in its entirety.

### FIELD

The present invention relates to a surgical rod cutter. It is suitable for and intended for mechanically severing surgical rods, which are used for example for stabilizing or fixing bones or parts of bones and which normally consist of materials of high strength and high hardness, such as titanium alloys or cobalt chromium alloys.

### BACKGROUND

Devices for mechanically severing surgical rods are generally known. They serve to allow surgical staff to cut or sever surgical rods of different diameters and materials in an operating room and during an operation conveniently and without a major expenditure of force. Known devices are often non-ergonomic in view of their structural design. Some devices are operated by means of two levers with handle elements, said levers being connected by means of one or a plurality of hinges. This is disadvantageous in specific situations, for example if not both hands are free for operating the levers in this way. In addition, depending on the dimensions and the material of the rod to be severed and the mechanism of the rod cutter, a comparatively high expenditure of force may be necessary for severing the rod, and this necessitates physically demanding work on the part of the user in the operating room. For preventing the severing forces to be applied from becoming unacceptably high, comparatively long levers have to be provided, and this makes the device bulky and difficult to handle.

Another drawback of known rod cutters may be their limited suitability for reprocessing. Depending on the respective structural design, it is, for example in view of long levers, necessary to disassemble the rod cutter at least partially, such disassembling being, however, difficult. The effort required for reprocessing may therefore be disadvantageously high.

A further drawback of known rod cutters may be seen in that their cutting edges, which are used for severing the normally very hard surgical rods, become rapidly blunt. This leads to the necessity of using high-grade and expensive materials or to a reduction of the quality of the cut or severed edges produced. Reworking of the cutting edges is normally not possible or only possible to a limited extent.

U.S. Pat. No. 5,836,937 discloses a surgical cutter after the fashion of a bolt cutter comprising two cutting jaws connected to two handles via a lever mechanism. The lever mechanism and the handles provide a transmission ratio of such a nature that surgical rods can be to severed manually. In the area of the cutting edges, a U-shaped guide for the rod to be severed is provided, by means of which injuries of bones or tissue during severing of the rod are largely prevented. The device is disadvantageous insofar as it requires operation with both hands and, depending on the material and the diameter of the rod to be severed, comparatively high operating forces. Reprocessing seems to be problematic in view of the numerous narrow gaps in the area of the leverage mechanism, the non-existing possibility for simple disassembly and the long levers required for accomplishing a suitable transmission ratio.

U.S. Pat. No. 5,988,027 discloses a manually operable surgical rod cutter comprising a supporting base, a cutting tool arranged on said base and a handle for operating the cutting tool. The cutting tool essentially comprises two cylindrical adjacent cutting components whose cylinder axes are in alignment with one another. The cutting components are adapted to be rotated relative to one another about the respective cylinder axis. The first cutting component is connected to the supporting base and the second cutting component is connected to the handle, so that the cutting components are rotated relative to one another when the handle is pivoted. The cutting components have each formed therein bore holes, which are eccentric with respect to the respective cylinder axis and in which a rod to be severed is to be arranged. When the rod cutter is operated, the bore holes of the two cutting components will be displaced relative to one another in accordance with the eccentricity, whereby a rod inserted in these bore holes will be sheared off The handle is extensible, so that the effective leverage and, consequently, the cutting force applied can be varied. U.S. Pat. No. 5,261,303 discloses a similar rod cutter whose handle is foldable. These rod cutters can be operated with one hand, but, to this end, it is necessary to fixedly mount the rod cutter on a table or the like. Also their reprocessing is problematic for the above-mentioned reasons. Simple disassembly is not possible.

U.S. Pat. No. 6,058,820 discloses a surgical rod cutter with a supporting base, a handle rotatably arranged thereon and two cutting arms. One cutting arm has its first end pivotably articulated on the supporting base, whereas the other cutting arm has its first end pivotably connected to the handle. The two cutting arms are connected at the respective other end thereof by means of a stud bolt, so that they can be pivoted relative to one another about a common pivot axis. The pivot arms have throughholes, which define cutting edges and into which a rod to be severed can be inserted. By operating the handle, the two cutting arms are pivoted relative to one another, whereby the throughholes are displaced relative to one another and a rod accommodated therein is sheared off. In view of its structural design and the numerous narrow spaces between parts that are moved relative to one another, this rod cutter is difficult to sterilize. Simple disassembly is not possible.

U.S. Pat. No. 8,127,454 discloses a surgical rod cutter as well as a method of determining the length of a rod piece severed by means of said rod cutter. The rod cutter has two handles, the end of each of said handles having arranged thereon a cutting element of a tool head by means of a polygonal reception means. The cutting elements are held by means of a cap such that they are rotatable relative to one another and they each have provided therein bores with cutting edges into which a rod to be severed is inserted. The handles are telescopically extensible. With the aid of a clip that can be arranged on the rod to be severed, the length of the piece to be cut off can be determined precisely. This rod cutter is disadvantageous insofar as it has to be operated with both hands.

US 2008/0000091 A1 discloses a tool for cutting through threaded rods. This tool has a supporting base on which two cutting plates are adapted to be rotated about a common axis relative to one another and relative to the supporting base. The cutting plates abut on one another and their circumference has formed therein recesses with cutting edges. The recesses have inserted therein a threaded rod to be cut through. Each of the cutting plates is provided with a lever, with which one of the cutting plates is articulated on the supporting base and the other cutting plate is articulated on a lever that is pivotably supported on the supporting base. When the lever is operated, the cutting plates are rotated relative to one another, so that the cutting edges are displaced relative to one another and a threaded rod accommodated in the recesses is sheared off. The supporting base is held by means of an additional handle. It is disadvantageous that the tool has to be operated with both hands. In view of the numerous narrow spaces in the area of the leverage mechanism and the cutting plates, reprocessing is problematic, if not impossible.

### SUMMARY

It is the object of the present invention to provide a device by means of which the process of severing, in particular intraoperative severing, of surgical rods in an operating room is simplified and the expenditure of force required is reduced. The device should preferably be handy, user-optimized and ergonomic, and it should allow reprocessing, i.e. cleaning (optionally sterilizing), without major effort. Last but not least, a high quality of the cutting edges should preferably be guaranteed even after a prolonged period of use.

According to the present invention, the above object is achieved by a surgical rod cutter having a first housing-shaped device part and a second rotary device part. The device parts are supported relative to one another and adapted to be rotated relative to one another about an axis of rotation. The first device part has formed therein a continuous axial opening for accommodating a rod to be severed. The opening has its longitudinal axis oriented in the direction of the axis of rotation and, at the end side thereof, a cutting edge. The second device part has formed therein a continuous axial opening for accommodating the rod to be severed, said opening having its longitudinal axis oriented in the direction of the axis of rotation and, at the end side thereof, a cutting edge which is formed in opposed relationship with the cutting edge of the first device part. The opening of the first device part and the opening of the second device part are arranged with the same amount of eccentricity with respect to the axis of rotation. The first device part and the second device part are operatively connected to one another by means of a tooth system and are adapted to be positioned relative to one another.

The object is especially achieved by a surgical rod cutter having a first device part and a second device part, said device parts being supported relative to one another and adapted to be rotated relative to one another about an axis of rotation, wherein the first device part has formed therein a continuous opening for accommodating a rod to be severed, said opening having its longitudinal axis oriented in the direction of the axis of rotation and, at the end side thereof, a cutting edge, wherein the second device part has formed therein a continuous opening for accommodating the rod to be severed, said opening having its longitudinal axis oriented in the direction of the axis of rotation and, at the end side thereof, a cutting edge which is formed in opposed relationship with the cutting edge of the first device part, wherein the opening of the first device part and the opening of the second device part are arranged with the same amount of eccentricity with respect to the axis of rotation, and wherein the first device part and the second device part are operatively connected to one another by means of teeth and are adapted to be positioned relative to one another.

The object is additionally achieved by a surgical rod cutter comprising a first device part and a second device part, said device parts being supported relative to one another and adapted to be rotated relative to one another about an axis of rotation, wherein the first device part has formed therein a continuous opening for accommodating a rod to be severed, said opening having its longitudinal axis oriented in the direction of the axis of rotation and, at the end side thereof, a cutting edge, wherein the second device part has formed therein a continuous opening for accommodating the rod to be severed, said opening having its longitudinal axis oriented in the direction of the axis of rotation and, at the end side thereof, a cutting edge which is formed in opposed relationship with the cutting edge of the first device part, wherein the opening of the first device part and the opening of the second device part are arranged with the same amount of eccentricity with respect to the axis of rotation, wherein at least one of said openings of the first device part and of the second device part has replaceably arranged therein a bushing provided with said cutting or severing edge. A special advantage is here achieved when the opening of the first as well as that of the second device part have each arranged therein such a bushing.

In other words, the surgical rod cutter according to the present invention comprises a preferably cylindrical housing with an axially extending cylindrical blind bore, which is followed in the axial direction by a first rod-accommodating throughhole that is axially displaced to a specific extent relative to the center axis of the cylindrical blind bore, i.e. eccentrically positioned. In addition, the surgical rod cutter includes a shearing/rotary disk with an (external) tooth rim, a bearing pin, which is rotatably insertable/inserted in the cylindrical blind bore, extending axially from the center of said shearing/rotary disk. The bearing pin includes an axial second rod-accommodating throughhole, which is positioned eccentrically to the bearing pin axis, the axial displacement relative to the longitudinal axis of the housing corresponding substantially to that of the first rod-accommodating throughhole.

The diameter of the two throughholes is substantially equal, so that, at a specific relative angular position, the two rod-accommodating throughholes are congruent and so that they assume an increasingly intersecting position when the rotary disk is moved relative to the housing.

The end faces of the bearing pin and of the bottom of the cylindrical blind bore facing each other have provided thereon a cutting or shearing edge, preferably in the form of a (replaceable) insertion bushing inserted into the respective rod-accommodating throughhole. Last but not least, a gear which is adapted to be driven/driven manually or by means of a motor is supported in the housing, said gear being adapted to be operatively engaged or being operatively engaged with the tooth rim so as to apply a rotary driving force to the rotary disk with a high transmission ratio.

The device according to the present invention is particularly suitable for severing a surgical rod with little driving force due to the high transmission ratio between the tooth rim and the gear. This driving force can easily be applied by hand.

The first and the second device parts (housing, rotary disk) may each have provided therein a plurality of openings/throughholes for accommodating rods to be severed, in particular openings/throughholes with different diameters for severing rods of different thicknesses.

In the device according to the present invention, especially the openings/throughholes of the first and/or the second device part(s) may have replaceably arranged therein a respective bushing provided with the cutting or severing edge, as has been indicated hereinbefore. Thus the bushings, when worn, can be replaced by new bushings.

In addition, the first device part and the second device part of the device according to the present invention may be connected operatively by means of teeth and adapted to be positioned relative to one another, whereby transmission ratios can be realized that are higher than those of known leverage systems.

Since the openings/throughholes of the first and second device parts of the device according to the present invention are arranged with a substantially equal amount of eccentricity with respect to the axis of rotation, it is guaranteed that, when the two device parts occupy a specific (angular) position relative to one another, the openings/throughholes will be congruent, i.e. positioned one on top of the other, so that a rod can easily be inserted through both openings/throughholes.

Making use of the present invention, the cutting, shearing or severing force can be transmitted by means of a toothed gear unit to the surgical rod to be severed, whereby a plurality of advantages is achieved, for example that a motor or a motor system (having little driving power) can be connected to the device, so that the latter can be used not only manually but also in a driven mode. Another advantage is that, in comparison with rod cutters known from the prior art, in the case of which the necessary severing forces are accomplished through an effect produced by long levers, the toothed gear unit allows to provide a very compact device. On the basis of the toothed gear unit, the transmission ratio of the rod cutter can easily be determined or even varied, without this having necessarily an effect on the overall size of the device, as in the case of a lever mechanism according to the prior art.

When used in an operating room, the device according to the present invention requires little space and is therefore very versatile in use. The forces that have to be applied for severing the rod can advantageously be determined to be comparatively weak by selecting an adequate transmission ratio. Due to its compact structural design, the device can, in addition, be cleaned and sterilized in conventional reprocessing units. A special advantage is that the device according to the present invention can be operated by one hand.

According to a particularly advantageous embodiment, the cutting bushing may be a single-use product, said cutting bushing being preferably installed in the rod cutter for single use only. Nevertheless, the device according to the present invention also allows a cutting bushing to be used more than once. In the case of a single-use cutting bushing, insufficient quality of the severing edge caused by wear will always be avoided. A constant and high quality of the severing edges of the surgical rod is guaranteed. Since the cutting bushings are normally replaced after having been used once, tool costs can be kept low in comparison with the device according to the prior art. It is for example possible to use cutting bushings of lesser material quality without reducing the quality of the severed surfaces produced. According to a particularly preferred embodiment, cutting bushings made of cemented carbide or high-speed steel may be used (low-priced standard parts), whereas prior art devices necessitate the use of expensive special-purpose steels (in particular for the cutting edges) so as to guarantee a sufficient tool life and cut surfaces and edges of satisfactory quality. Since the cutting bushings according to the present invention have to fulfill lower demands as regards hardness and wear resistance, a greater variety of materials can be used and materials can be selected in a particularly advantageous manner primarily with respect to other characteristics, such as biocompatibility or costs.

A replaceable cutting bushing is advantageous insofar as, after having been used, it can be replaced and, if desired, reworked and reprocessed, for example ground, in a particularly easy manner. The cutting bushing preferably has a simple shape, it may for example have substantially the shape of a hollow cylinder with a circumferentially extending collar on one end. The end located opposite the collar has the cutting or severing edge of the bushing formed thereon. According to a specially preferred embodiment, the cutting-edge-side end face of the bushing is oriented at right angles to the longitudinal axis of the bushing and can thus easily be produced and, if necessary, reworked, in particular ground. The bushing may abut on a shoulder in the continuous opening with its circumferentially extending collar. It is preferably tensioned against said shoulder so that simple mounting as well as a precisely defined position of a cutting bushing will be guaranteed. According to one embodiment, the cutting bushing is secured in position and/or positioned in the opening of the first and second device part, respectively, by means of a screw, in particular a manually operable screw. The cutting bushing can thus be replaced easily, rapidly and at a reasonable price in situ in the operating room.

According to an embodiment of the present invention of the type that has already been indicated hereinbefore, the first device part defines a housing and may especially be provided with a support surface for positioning on a suitable support. It will be of advantage to configure the support surface as an anti-skid surface, for example by means of an adequate coating or surface structuring. Alternatively or additionally, it may be provided with one or a plurality of vacuum cups, which can be evacuated and which thus allow the device to be fixedly positioned on a smooth surface without permanent fastening. The housing may, however, also be configured for permanent fastening to a suitable support, such as a side table or the like. To this end, it may for example be provided with holes by means of which the device can be fixed on the support by screws.

According to another optional embodiment of the device, the first device part, especially the blind bore thereof, has arranged/inserted therein a bearing, preferably a needle bearing, for taking up radial forces. The bearing consists preferably of a corrosion-resistant material of sufficiently high strength. According to a preferred embodiment of the invention, the bearing is a dry running bearing which does not necessitate lubrication, a circumstance which is particularly advantageous for use in the field of medicine. In addition, the bearing runs very easily and has only low friction values, the force required for operating the device being thus very small. An example for a particularly advantageous material for the bearing rollers is the material having the (DIN) standard designation 1.4125. This exemplary material allows unproblematic reprocessing of the complete bearing area.

According to another preferred embodiment, the teeth are formed on the second device part, preferably in the circumferential direction, at least sectionwise. The teeth may either be internal or external teeth. Preferably, the first device part has formed thereon or therein a reception means for a setting element and a gear, respectively, which—when arranged in said reception means—is engagement with the teeth. The setting element may be manually operable, i.e. it may for example be configured in the form of a setting wrench with a gear shaft and a manual operating handle, or it may be connected to a drive motor or a drive transmission so as to allow a motor-driven operation of the device.

According to a specially preferred embodiment, the device may comprise a locking means, which allows rotation of the second device part in only one direction. Preferably, this locking means consists essentially of a spring-biased ratchet, which cooperates with the teeth and which allows rotation of the second device part in a first direction of rotation, but prevents rotation in the opposite direction of rotation. The locking means can be manually operated and/or unlocked.

The first device part may have formed therein a rinsing passage. This rinsing passage preferably leads from the bearing to a connection, preferably a Luer lock connection, and serves to supply a cleaning agent in a particularly easy manner to the interior of the device and especially to the bearing in the course of a cleaning and reprocessing (sterilizing) operation. Such a device can advantageously be cleaned in a particularly easy manner.

Taking all this into account, the device according to the present invention is handy and ergonomic and can be positioned at various sites in a stable manner. The device consists preferably of a material having the (DIN) standard designation 1.4034 (or X46Cr13).

### BRIEF DESCRIPTION OF THE DRAWING FIGURES

Additional features and advantages of the present invention can be seen from the following exemplary description of specially preferred embodiments that makes reference to the figures, in which:

FIG. 1 shows a schematic representation of the surgical rod cutter in a front view;

FIG. 2 shows a schematic representation of the device according to FIG. 1 in a sectional view along line 1-1 in FIG. 1;

FIG. 3 shows a schematic representation of the device according to FIGS. 1 and 2 in a top view;

FIG. 4 shows a detail of the bearing of the device according to FIG. 2;

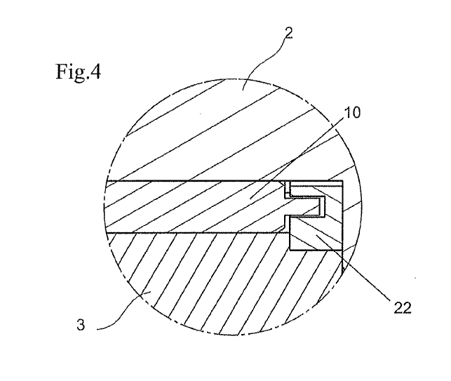

FIG. 5 shows a schematic representation in which a bushing having a cutting, shearing or severing edge is installed in the first device part used as a housing;

FIG. 6 shows a schematic representation in which a bushing having a cutting, shearing or severing edge is installed in the second device part;

FIG. 7 shows a schematic representation in which the second device part is installed in the first device part;

FIG. 8 shows a schematic representation in which a surgical rod to be severed is inserted in the device; and

FIG. 9 shows how the device is operated for severing the surgical rod.

### DETAILED DESCRIPTION

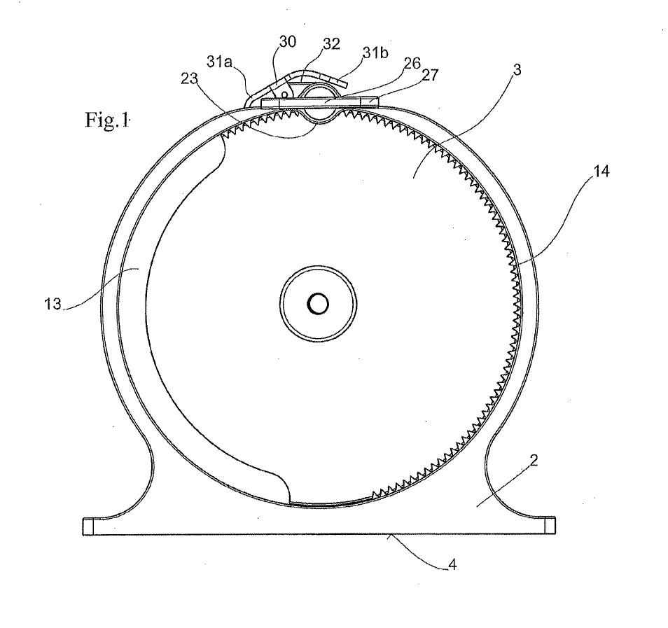

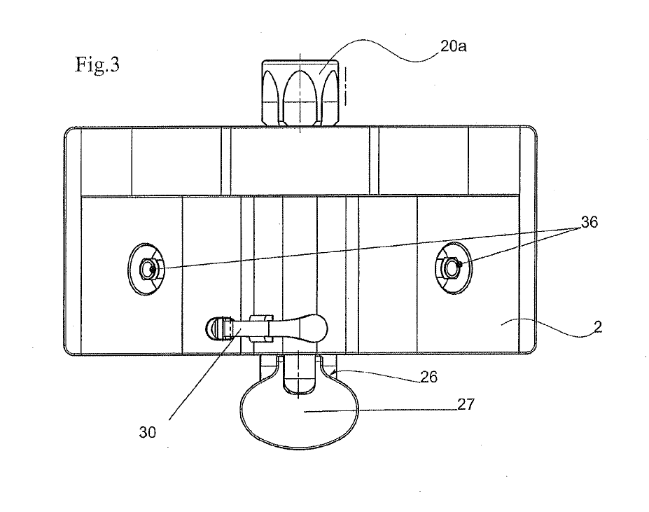

FIGS. 1 and 3 show a surgical rod cutter 1. It comprises a body 2 as a first device part/housing as well as a shearing disk 3 as a second device part. The body 2 as well as the shearing disk 3 consist of a highly hardened and tempered stainless steel, for example X46Cr13 (or also 1.4034), a circumstance that guarantees, firstly, sufficient strength and, secondly, the possibility of reprocessing, i.e. cleaning and sterilizing, the device without any formation of corrosion.

The body 2 is substantially cylindrical in shape and its outer circumferential surface has provided thereon a skid-proof support surface 4 by means of which it can be positioned stably and safely on a suitable working surface in an operating room, for example on a side table or the like. The body 2 has formed therein an axially continuous opening or throughhole 5 whose longitudinal axis extends substantially horizontally when the body 2 is positioned on a horizontal support surface 4. The throughhole 5 has in an axial longitudinal center section A thereof a small diameter d (rod-reception/bushing-reception throughhole) and is expanded to a medium diameter D₁ in an axial longitudinal end section B. At its longitudinal end section C, which is located opposite said section B, it is expanded to a diameter D₂ (cylindrical blind bore). The transition from section B to section A is configured as a shoulder 6 and the transition from section C to section A is configured as a shoulder 7. Section B is provided with a female thread 8. Section C is configured as a bearing seat 9 for a needle bearing 10. On the side of section C located opposite to section A, the throughhole 5 is expanded to a cylindrical reception means 11 for the shearing disk 3.

The shearing disk 3 is provided with a bearing section/bearing pin 12 for the needle bearing 10, said bearing pin 12 extending (integrally) from the shearing disk 3 centrally and perpendicular thereto. In a section of its circumference, the shearing disk 3 is provided with a (circumferential) recess 13. In the remaining part of its circumference, it is has external teeth 14 provided thereon. The shearing disk 3 has a throughhole 15 having in section D the diameter d and in section E the diameter D₁. The transition from section D to section E is configured as a shoulder 16. Section E is provided with a female thread 17. In addition, the throughhole 15 is positioned out of center and is displaced relative to the center axis of the shearing disk 3 to a predetermined extent, as will be described in more detail hereinbelow.

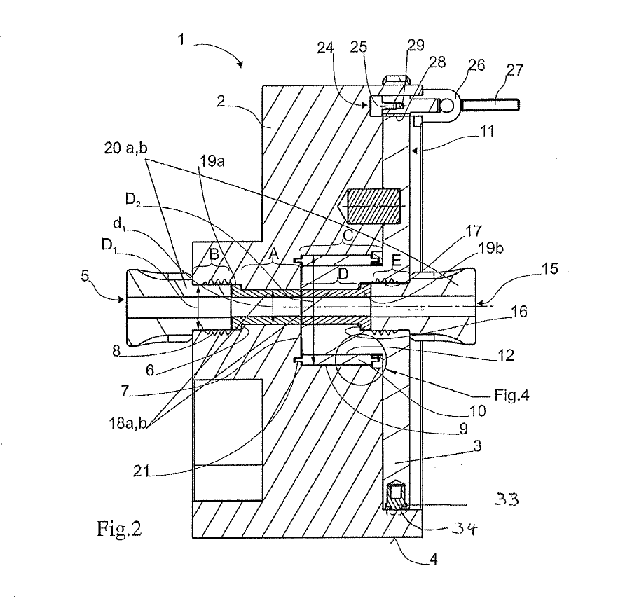

A cutting bushing 18a is arranged/inserted/insertable in the throughhole 5 of the body 2, whereas a (counter) cutting bushing 18b is arranged/inserted/insertable in the throughhole 15 of the shearing disk 3. The cutting bushings 18a and 18b are identical components comprising a section with the diameter d and a stepped portion 19a and 19b, respectively, with the diameter D1, so that, as shown in FIG. 2, their longitudinal section is substantially T-shaped. The cutting bushing 18a is secured in position by means of a retaining screw 20a, which is screwed into the female thread 8 of the body 2, so that its stepped portion 19a abuts on the shoulder 6. The cutting bushing 18b is secured in position by means of a retaining screw 20b, which is screwed into the female thread 17 of the shearing disk 3, so that its stepped portion 19b abuts on the shoulder 16. The cutting bushings 18a and 18b consist of biocompatible cemented carbide and are configured as single-use components, i.e. they are intended to be used only once.

The shearing disk 3 is supported in the body 2 by means of the needle bearing 10 such that it is rotatable relative to said body 2 about the axis of rotation of the needle bearing 10. FIG. 2 shows that, in section C of the throughhole 5 of the body, the bearing seat 9 is displaced by an eccentricity e relative to the center axis of sections A and B. The throughhole 15 of the shearing disk 3 is displaced relative to the bearing section 12 by the same eccentricity e. At the position which the body 2 and the shearing disk 3 occupy relative to one another and which is shown in FIG. 2, the center axes of sections A, B, D and E—and consequently also the center axes of the cutting bushings 18a and 18b—are congruent.

The needle bearing 10 is secured in position on its left side, when seen in FIG. 2, in a recess 21 in the shoulder 7 and on its right side, when seen in FIG. 2, by means of a bearing ring 22. The recess 21 defines the fixed bearing and the bearing ring 22 defines the floating bearing, as can especially be seen from the detailed representation of the figure. The bearing ring 22 is secured in position in the body, for example by clamping, screw-fastening or welding. The needle bearing 10 comprises bearing rollers made of the material “KCMO”.

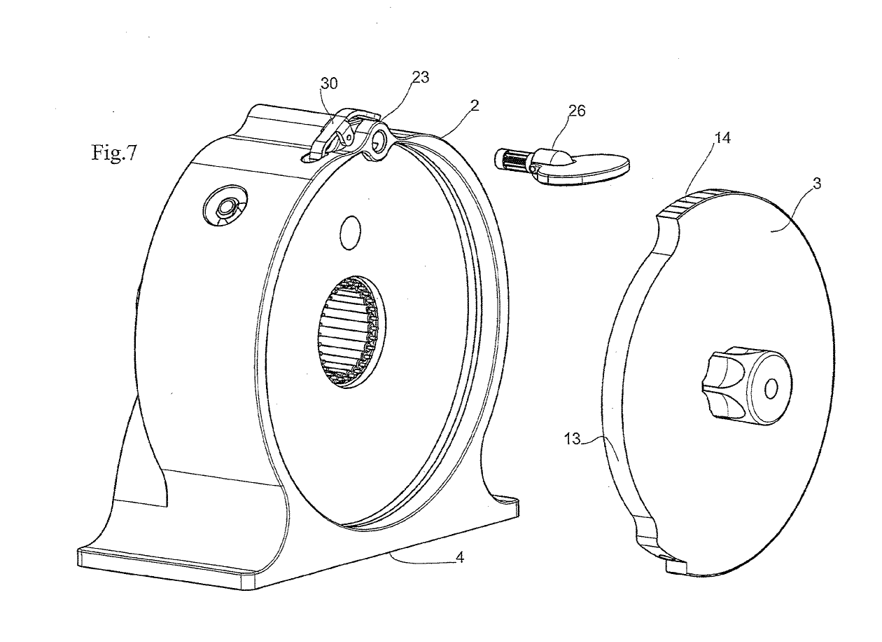

On its side located on the upper right in FIG. 2, the body 2 is provided with an eye or ear 23. A recess/bore 24 is formed on the bottom of the reception means 11 in opposed relationship with the eye/ear 23, said recess/bore 24 having arranged therein a bearing pin 25. This bearing pin 25 serves as a bearing for a gear-type setting wrench 26, which is used as a setting element and by means of which an operator can operate the rod cutter 1/the shearing disk 3. The setting wrench 26 is shown in a perspective view in FIG. 7 prior to insertion in the body 2, and in FIG. 2 in the installed condition in said body 2. It comprises a handle (winged nut) 27, external teeth 28 as well as a bearing opening 29. In the installed condition, the bearing pin 25 engages the bearing opening 29, so that the setting wrench 26 is guided at its free end, which is the left end in FIG. 2. On the right side, it is guided via the eye 23. The external teeth 28 of the setting wrench 26 are in engagement with the external teeth 14 of the shearing disk 3, so that, when the setting wrench 26 is rotated by an operator, the shearing disk 3 will rotate about the axis of rotation of the needle bearing 10.

In order to prevent inadvertent resetting of the shearing disk 3 in the opposite direction, for example due to the elasticity of the rod to be cut/severed, the rod cutter 1 comprises a locking means in the form of a catch mechanism comprising a ratchet 30 (cf. in particular FIGS. 1, 3 and 7).

This ratchet 30 comprises two locking arms 31a,b and is biased by means of a spring 32 towards a position at which the shearing disk 3 is locked. One of the locking arms—in FIG. 1 the left locking arm 31a—is in engagement with the external teeth 14 of the shearing disk 3. Via the other locking arm 31b, the catch mechanism can be manually released by an operator. When the shearing disk 3 is rotated (in FIG. 1 anticlockwise) by means of the setting wrench 26 during a severing operation, the catch mechanism will slip. In the case of a rotation in the opposite direction, the locking arm 31 a will lock due to engagement with the external teeth 14.

A portion of the circumference of the shearing disk 3 between the circumferential recess 13 and the external teeth 14 has formed therein a radial bore having arranged therein a ball pressure element 33 that is biased by means of a spring that is not shown. On its side facing radially outwards with respect to the shearing disk 3, this ball pressure element is partially spherical and engages a circumferentially extending guide track 34 formed in the body 2. The guide track 34 has formed therein a recess, which is not shown in the figures and with which the ball pressure element 33 engages with a sound audible to the operator at the “zero position” shown in FIG. 2, at which the center axes of sections A and D of the throughholes 5 and 15 are in alignment with one another. At this “zero position” a rod 35 to be severed can be inserted into the cutting bushings 18a,b.

The body 2 has formed therein fluid passages that are not shown in the figures. These fluid passages are provided with Luer lock connections 36 at their openings towards the outside of the body 2 and they define a flow connection to section C of throughhole 5 of the body 2. Through these fluid passages the needle bearing 10 can be rinsed with cleaning and sterilization fluids during reprocessing.

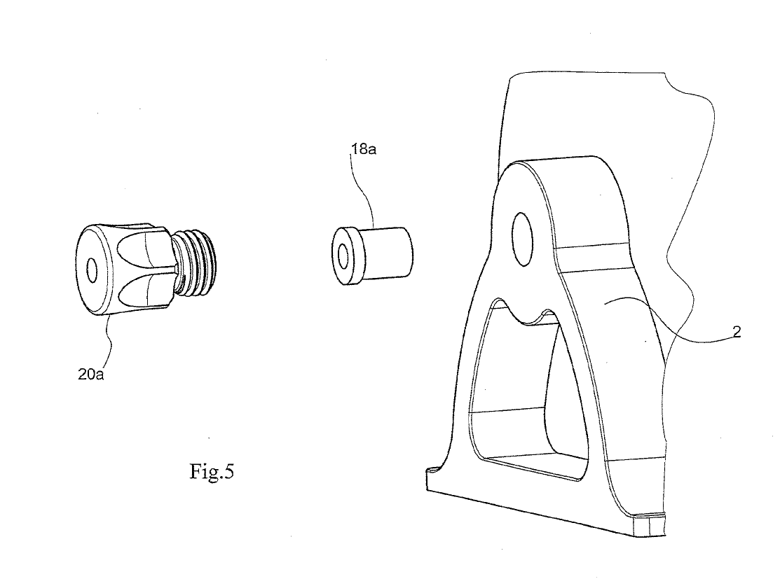

The function of the rod cutter 1 is explained with respect to FIGS. 5 to 9.

FIG. 5 shows how the cutting bushing 18a is installed in the body 2. The cutting bushing 18a is inserted, from the side constituting the left side in FIG. 2, into section A of the throughhole 5 and is there pressed against the shoulder 6 and secured in position by screwing the retaining screw 20a into the female thread 8. Due to the fact that the cutting bushing 18a abuts on said shoulder 6, the position of the cutting, severing or shearing edge of said cutting bushing 18a is precisely defined.

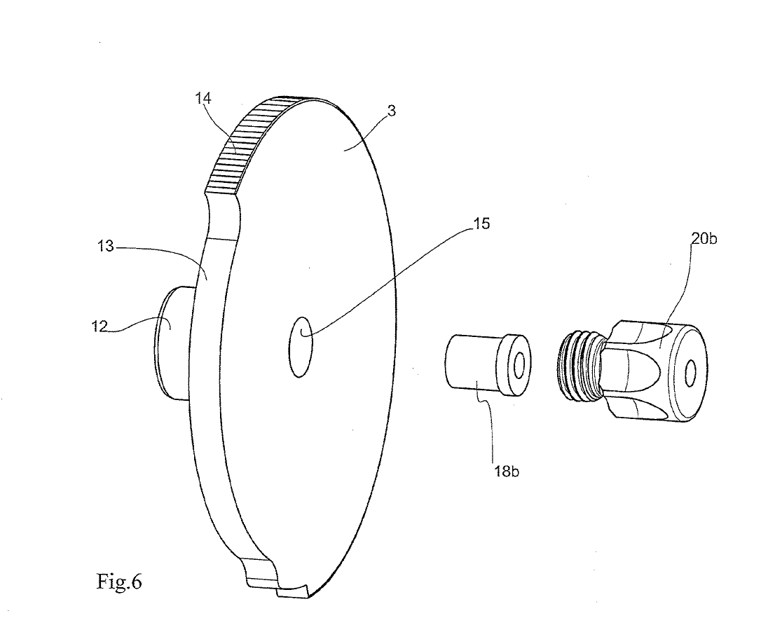

FIG. 6 shows how the cutting bushing 18b is installed in the shearing disk 3. The cutting bushing 18b is inserted, from the side constituting the right side in FIG. 2, into section D of the throughhole 15 and is there pressed against the shoulder 16 and secured in position by screwing the retaining screw 20b into the female thread 17. Due to the fact that the cutting bushing 18b abuts on said shoulder 16, the position of the cutting, severing or shearing edge of said cutting bushing 18b is precisely defined.

FIG. 7 shows how the shearing disk 3, which has the cutting bushing 18b and the retaining screw 20b mounted therein, is installed in the body 2. The shearing disk 3 is oriented relative to the body 2 such that its circumferential recess 13 is located in the area of the eye 23. During installation, the bearing section 12 of the shearing disk 3 is inserted into the needle bearing 10 until the cutting, severing or shearing edges of the two cutting bushings 18a,b have reached their respective intended end position, for example a position at which they abut on one another or are separated from one another by a gap of small width. At this position the ball pressure element 33 lockingly engages the guide track 34. Subsequently, the setting wrench 26 is inserted into the eye 23 provided for this purpose. Making use of the setting wrench 26, the shearing disk 3 is rotated anticlockwise until the external tooth rim 14 is moved to a position behind the eye 23 thus blocking an axial displacement of the shearing disk 3. This is possible in spite of the ratchet mechanism, since the circumferential recess 13 is located in the area of the ratchet 30 which is not in engagement with the external teeth 14. The shearing disk 3 is rotated until the “zero position” shown in FIG. 2 has been reached. When this “zero position” has been reached, the ball pressure element 33 lockingly engages, with an audible sound, the above-mentioned recess in the guide track 34, thus indicating to the operator that the “zero position” has been reached.

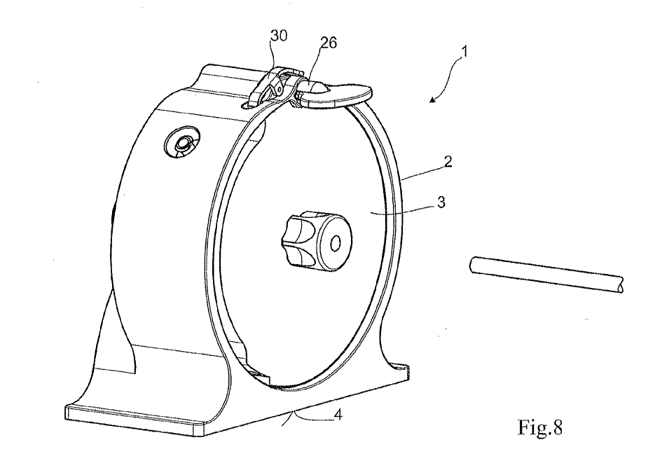

Subsequently, the rod 35 to be severed is inserted into the rod cutter 1 as shown in FIG. 8. The rod 35 can only be inserted in the “zero position”, since this is the only position of the shearing disk 3 relative to the body 2 at which the cutting bushings 18a,b are in alignment.

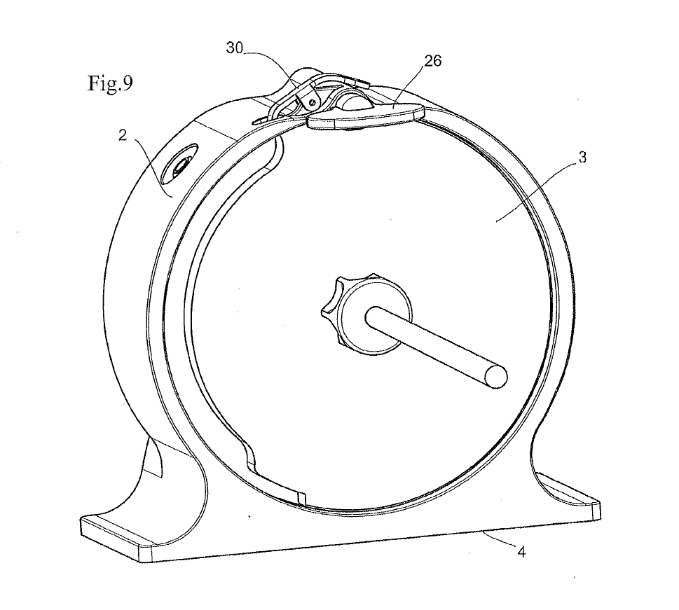

FIG. 9 shows how the rod 35 is severed by rotating the shearing disk 3 still further by operating the setting wrench 26. Due to the above described eccentricities of the needle bearing 10 relative to section A of throughhole 5—and consequently to the cutting bushing 18a—as well as relative to section D of throughhole 15—and consequently to the cutting bushing 18b—a rotation of the shearing disk starting at the “zero position” results in a circular movement (circulating movement) of the cutting bushing 18b with a radius corresponding to twice the eccentricity about the center axis of the needle bearing 10 relative to the cutting bushing 18a. This relative movement has the effect that the rod 35 inserted in the cutting bushings 18a and 18b is sheared off.

The operating force required for shearing off the inserted rod remains, however, so small that it can easily be applied by hand. This is accomplished, on the one hand, by the transmission ratio between the setting wrench and the external tooth rim on the shearing disk 3 and, on the other hand, by the transmission ratio between the rotation distance covered by the shearing disk 3 and the eccentric distance covered by the cutting bushings 18a,b.

1.-11. (canceled)

12. A surgical rod cutter comprising:
a first housing-shaped device part and a second rotary device part, said device parts being supported relative to one another and adapted to be rotated relative to one another about an axis of rotation,
the first device part having formed therein a continuous axial opening for accommodating a rod to be severed, said opening of the first device part having its longitudinal axis oriented in the direction of the axis of rotation and, at the end side thereof, a cutting edge,
the second device part having formed therein a continuous axial opening for accommodating the rod to be severed, said opening of the second device part having its longitudinal axis oriented in the direction of the axis of rotation and, at the end side thereof, a cutting edge which is formed in opposed relationship with the cutting edge of the first device part,
the opening of the first device part and the opening of the second device part being arranged with the same amount of eccentricity with respect to the axis of rotation,
the first device part and the second device part being operatively connected to one another by means of a tooth system, the first device part and the second device part being adapted to be positioned relative to one another.

13. The surgical rod cutter according to claim 12, wherein the first device part defines a substantially cylindrical housing provided with a support surface for positioning on a support.

14. The surgical rod cutter according to claim 12, wherein the opening of the first device part has arranged therein an inner bearing in the form of a needle bearing.

15. The surgical rod cutter according to claim 12, wherein the second device part is provided with internal or external teeth, which are formed at least sectionwise in a circumferential direction.

16. The surgical rod cutter according to claim 15, wherein the first device part has formed therein a reception means for a setting element that is in mesh with the teeth of the second device part.

17. The surgical rod cutter according to claim 15, further comprising a locking means in the form of a ratchet, which cooperates with said teeth and which allows rotation of the second device part in a first direction of rotation, but prevents rotation in the opposite direction of rotation.

18. The surgical rod cutter according to claim 14, wherein the first device part has formed therein rinsing passages, which lead from the bearing to a Luer lock connection so as to supply a sterilizing agent to said bearing.

19. The surgical rod cutter according to claim 12, further comprising at least one bushing, the at least one bushing comprising a cemented carbide made of a biocompatible cemented carbide.

20. The surgical rod cutter according to claim 19, wherein the at least one bushing abuts on a shoulder in at least one of the openings, and is tensioned against said shoulder by means of a screw element.

21. A surgical rod cutter comprising:
a first housing-shaped device part and a second rotary device part, said device parts being supported relative to one another and adapted to be rotated relative to one another about an axis of rotation,
the first device part having formed therein a continuous axial opening for accommodating a rod to be severed, the opening of the first device part having its longitudinal axis oriented in the direction of the axis of rotation and, at the end side thereof, a cutting edge,
the second device part having formed therein a continuous axial opening for accommodating the rod to be severed, the opening of the second device part having its longitudinal axis oriented in the direction of the axis of rotation and, at the end side thereof, a cutting edge which is formed in opposed relationship with the cutting edge of the first device part,
the opening of the first device part and the opening of the second device part being arranged with the same amount of eccentricity with respect to the axis of rotation,
at least one of the opening of the first device part and the opening of the second device part having replaceably arranged therein a bushing provided with said cutting edge, said bushing being the single-use type.

22. The surgical rod cutter according to claim 21, wherein the first device part defines a substantially cylindrical housing provided with a support surface for positioning on a support.

23. The surgical rod cutter according to claim 21, wherein the opening of the first device part has arranged therein an inner bearing in the form of a needle bearing.

24. The surgical rod cutter according to claim 21, wherein the second device part is provided with internal or external teeth, which are formed at least sectionwise in a circumferential direction.

25. The surgical rod cutter according to claim 24, wherein the first device part has formed therein a reception means for a setting element that is in mesh with the teeth of the second device part.

26. The surgical rod cutter according to claim 24, further comprising a locking means in the form of a ratchet, which cooperates with said teeth and which allows rotation of the second device part in a first direction of rotation, but prevents rotation in the opposite direction of rotation.

27. The surgical rod cutter according to claim 23, wherein the first device part has formed therein rinsing passages, which lead from the bearing to a Luer lock connection so as to supply a sterilizing agent to said bearing.

28. The surgical rod cutter according to claim 21, wherein said bushing comprises a cemented carbide made of a biocompatible cemented carbide.

29. The surgical rod cutter according to claim 21, wherein said bushing abuts on a shoulder in at least one of the openings, and is tensioned against said shoulder by means of a screw element.

30. A surgical rod cutter comprising:
a first housing-shaped device part and a second rotary device part, said device parts being supported relative to one another and adapted to be rotated relative to one another about an axis of rotation,
the first device part having formed therein a continuous axial opening for accommodating a rod to be severed, said opening of the first device part having its longitudinal axis oriented in the direction of the axis of rotation and, at the end side thereof, a cutting edge,
the second device part having formed therein a continuous axial opening for accommodating the rod to be severed, said opening of the second device part having its longitudinal axis oriented in the direction of the axis of rotation and, at the end side thereof, a cutting edge which is formed in opposed relationship with the cutting edge of the first device part,
the opening of the first device part and the opening of the second device part being arranged with the same amount of eccentricity with respect to the axis of rotation,
the first device part and the second device part being operatively connected to one another by means of a tooth system, the first device part and the second device part being adapted to be positioned relative to one another,
at least one of the opening of the first device part and the opening of the second device part having replaceably arranged therein a bushing provided with said cutting edge, said bushing being the single-use type.
